(19) United States
(12) Patent Application Publication
(10) Pub. No.: US 2022/0008698 A1
(43) Pub. Date: Jan. 13, 2022

(54) AUTOMATIC EVALUATION OF A FILLING VOLUME OF AN OESOPHAGEAL BALLOON CATHETER

(71) Applicant: MAQUET CRITICAL CARE AB, SOLNA (SE)

(72) Inventor: Magnus HALLBÄCK, Danderyd (SE)

(21) Appl. No.: 17/310,152

(22) PCT Filed: Feb. 20, 2019
(86) PCT No.: PCT/SE2019/050155

§ 371 Date: Jul. 21, 2021

(51) Int. Cl. A61M 25/10; A61M 16/04; G16H 40/20; G16H 20/40; G16H 50/70; G16H 40/40

(52) U.S. Cl. CPC A61M 25/10184; A61M 16/0409; G16H 40/20; A61M 2016/0027; G16H 50/70; G16H 40/40; G16H 20/40

(57) ABSTRACT

The present disclosure relates to a method for automatic evaluation of a filling volume of an oesophageal balloon catheter (26) inserted into a mechanically ventilated patient (3). The method comprises obtaining (S3-S4) samples of an airway pressure, P_{aw}, and an oesophageal pressure, Pₑₛ, of the patient during an occlusion period in which respiration of the patient is prevented, evaluating (S5) the filling volume of the oesophageal balloon catheter by determining a ratio, ΔPₑₛ/ΔP_{aw}, between Pₑₛ and P_{aw} from a regression analysis of the Pₑₛ and P_{aw} samples, and communicating (S6) a result of the evaluation to a user.

### TECHNICAL FIELD

The present disclosure relates to use of an oesophageal balloon catheter for measuring an oesophageal pressure of a patient and, in particular, to a method, a computer program and a system for automatic evaluation of a filling volume of an oesophageal balloon catheter.

### BACKGROUND

The use of oesophageal balloon catheters for measurement of oesophageal pressure (Pₑₛ) as a surrogate for pleural pressure is a well-known technique, albeit not yet widely used among intensive care clinicians. However, with the development of new high-accuracy oesophageal balloon catheters, and several studies showing that such oesophageal balloon catheters may be used to accurately determine the oesophageal pressure of a subject, oesophageal balloon catheters have been rediscovered as clinically useful means for monitoring important aspects of the pulmonary mechanics of mechanically ventilated patients.

Pₑₛ may in itself be a useful diagnostic parameter in the assessment of the pulmonary mechanics of the patient. Most often, however, Pₑₛ measurements are used in conjunction with measurements of the airway pressure (P_{aw}) of the patient in order to calculate an estimate of the patient's transpulmonary pressure (Pₜₚ). In mechanical ventilation, the settings of the breathing apparatus may then be adapted to the estimated Pₜₚ in order to optimize lung recruitment manoeuvres and protective ventilation strategies.

A challenge in accurate determination of Pₑₛ is the handling and use of the oesophageal balloon catheter. The oesophageal balloon catheter is filled with a fluid, normally air, and correct filling volume and positioning of the balloon catheter within the oesophagus of the patient are of uttermost importance to obtain accurate Pₑₛ measurements.

The filling volume of the oesophageal balloon catheter can be evaluated through a so called occlusion test. In passive patients having no spontaneous breathing activity, a positive pressure occlusion test according to which the chest of the patient is gently compressed by the clinician during an expiratory-hold manoeuvre (end-expiratory occlusion) can be performed. The pressure swings in Pₑₛ and P_{aw} caused by the compression of the chest are identified and compared, and, if they are substantially the same (i.e. if the ratio ΔPₑₛ/ΔP_{aw} is close to unity), the filling volume of the oesophageal balloon catheter is considered to be correct. In active patients having a spontaneous breathing activity, a Baydur occlusion test can be used instead of a positive pressure occlusion test to evaluate the filling volume of the oesophageal balloon catheter in a similar manner. In this case, the negative pressure swings in Pₑₛ and P_{aw} caused by spontaneous breathing attempts during an expiratory-hold manoeuvre can be identified and compared, and, if they are substantially the same (i.e. if the ratio ΔPₑₛ/ΔP_{aw} is close to unity), the filling volume of the oesophageal balloon catheter is considered to be correct.

How to use an oesophageal balloon catheter and the benefits of measuring Pₜₚ of a mechanically ventilated patient is further discussed e.g. in the white paper entitled “Transpulmonary pressure measurement—Benefit of measuring transpulmonary pressure in mechanically ventilated patients”, by Dr. Jean-Michel Arnal and Dr. Dominik Novotni, published online by Hamilton Medical on https://www.hamilton-medical.com/dam/jcr:d825a80f-cd5c-44bd-845f-7fa2b3056aeb/Transpulmonary-pressure-measurement-white-paper-en-ELO20150614S.02.pdf (2019 Jan. 21).

How to evaluate the filling volume of the oesophageal balloon catheter from the bedside of a mechanically ventilated patient is further discussed in an article by Dr. Jean-Michel Arnal, entitled “Bedside Tip: How to measure esophageal pressure correctly”, also published online by Hamilton Medical on https://www.hamilton-medical.com/en/News/Newsletter-articles/Article˜2018-10-19˜Bedside-Tip:-How-to-measure-esophaqeal-pressure-correctly˜5189d03b-e7b4-4eed-966e-2fae8f42a13a˜.html (2019 Jan. 21).

In accordance with common practice and the principles taught in the above mentioned article, a clinician that wants to evaluate the filling volume of the oesophageal balloon catheter has to rely on a visual comparison of signal curves representing Pₑₛ and P_{aw}, typically displayed on a display of the mechanical ventilator. In a best case scenario, the clinician is willing to take the time and effort to manually identify a minimum and maximum value of Pₑₛ and P_{aw} from the signal curves during the occlusion period, and to calculate the ratio ΔPₑₛ/ΔP_{aw} ((max Pₑₛ−min Pₑₛ)/(max P_{aw}−min P_{aw})) to verify that the ratio is close to unity. This is a non-trivial task since the quality of the Pₑₛ and P_{aw} signal curves may be poor, and since the signal curves are not normally displayed in a manner allowing the Pₑₛ and P_{aw} signal curves to be easily compared. Therefore, evaluation of the filling volume of the oesophageal balloon catheter is a cumbersome and time consuming task which, in practice, is nearly never performed during ongoing mechanical ventilation.

As understood from above, non-existing or improper evaluation of the filling volume of the oesophageal balloon catheter may result in undesired use of an oesophageal balloon catheter introducing errors in the determination of Pₑₛ and Pₜₚ of the ventilated patient. This may, in turn, result in improper adjustment of ventilator settings and, ultimately, in reduced patient safety.

### SUMMARY

It is an objective of the disclosure to improve patient safety during mechanical ventilation employing ventilation settings that are based on a measured oesophageal pressure of the ventilated patient.

It is a particular objective of the disclosure to minimize the risk of introducing errors in the determination of an oesophageal pressure of a mechanically ventilated patient.

It is yet another objective of the disclosure to facilitate use of an oesophageal balloon catheter for determination of an oesophageal pressure of a mechanically ventilated patient.

These and other objectives are achieved in accordance with the present disclosure by a method, a computer program and a system for automatic evaluation of a filling volume of an oesophageal balloon catheter, as defined by the appended claims.

According to an aspect of the disclosure, there is provided a method for automatic evaluation of a filling volume of an oesophageal balloon catheter inserted into a mechanically ventilated patient.

The method comprises the steps of obtaining samples of an airway pressure (Pa) and an oesophageal pressure (Pₑₛ) of the patient during an occlusion period in which respiration of the patient is prevented, evaluating the filling volume of the oesophageal balloon catheter by determining a ratio (ΔPₑₛ/ΔP_{aw}) between Pₑₛ and P_{aw} from a regression analysis of the obtained Pₑₛ and P_{aw} samples, and communicating a result of the evaluation to a user, e.g. to an operator of a breathing apparatus providing the mechanical ventilation to the patient.

By performing a regression analysis on a plurality of samples of P_{aw} and a plurality of samples of Pₑₛ obtained during the occlusion period, the ratio ΔPₑₛ/ΔP_{aw} can be automatically determined e.g. by a computer of the breathing apparatus providing the mechanical ventilation to the patient, or by a computer of a patient monitoring system for monitoring mechanically ventilated patients. The automated procedure minimizes manual workload, thereby allowing the clinician to focus on the patient and other clinical tasks, thus improving patient safety. Furthermore, the automated procedure enables the filling volume of the oesophageal balloon catheter to be properly evaluated in conjunction with determination of Pₑₛ, thus minimizing the risk of introducing errors in the determination of Pₑₛ and other parameters that are calculated based on Pₑₛ, such as the transpulmonary pressure (Pₜₚ) of the ventilated patient.

The result of the evaluation may comprise the determined ΔPₑₛ/ΔP_{aw} ratio and/or an indication on whether or not the filling volume of the oesophageal balloon catheter is acceptable, which indication is based on the determined ΔPₑₛ/ΔP_{aw} ratio. For example, the method may comprise the steps of determining, based on the ΔPₑₛ/ΔP_{aw} ratio, if the filling volume of the oesophageal balloon catheter is within a predetermined acceptance range, and communicating whether or not the filling volume of the oesophageal balloon catheter is within the acceptance range to the user. As discussed above, the ratio ΔPₑₛ/ΔP_{aw} should be close to unity if the filling volume of the oesophageal balloon catheter is correct. The predetermined acceptance range for the filling volume of the oesophageal balloon catheter may thus be defined in terms of a predetermined ratio acceptance range for the ΔPₑₛ/ΔP_{aw} ratio. The predetermined ratio acceptance range may, for instance, be 0.8-1.2.

The ΔPₑₛ/ΔP_{aw} ratio may be determined using any type of automated regression analysis for estimating a relationship between Pₑₛ and P_{aw}. For example, the ΔPₑₛ/ΔP_{aw} ratio may be determined as a slope of a curve resulting from the regression analysis, i.e. as the slope of a regression function estimated from the regression analysis. The regression analysis may be a linear regression analysis assuming a linear relationship between Pₑₛ and P_{aw}. In this case, the ΔPₑₛ/ΔP_{aw} ratio may be determined as the slope of the linear regression function resulting from the linear regression analysis.

The method may further comprise the steps of determining a quality measure of the evaluation based on a correlation between the Pₑₛ and P_{aw} samples, and communicating information indicative of an uncertainty in the evaluation of the filling volume of the oesophageal balloon catheter to the user, which information is based on the determined quality measure. This is advantageous in that the user can be provided with information relating to the reliability of the evaluation. The method may comprise the steps of automatically determining if the quality measure is within an acceptable quality range, and communicating an alert and/or a recommendation to repeat the automatic evaluation of the filling volume of the oesophageal balloon catheter to the user if the quality measure falls outside the acceptable quality range.

The quality measure may be any measure indicative of how well the regression predictions approximate the obtained Pₑₛ and P_{aw} samples. In one example, the quality measure may be the coefficient of determination (R²). That a quality measure indicative of the reliability of the evaluation of the filling volume is readily available from the regression analysis is another advantageous feature of the proposed procedure.

The method may further comprise the steps of determining a magnitude of change in Pₑₛ and/or P_{aw} during the occlusion period from the obtained Pₑₛ and P_{aw} samples, and communicating information comprising an alert and/or a recommendation to repeat the automatic evaluation of the filling volume of the oesophageal balloon catheter to the user if the magnitude of change in Pₑₛ and/or P_{aw} during the occlusion period is below a certain threshold value. This is advantageous in that the user can be alerted and/or prompted to repeat the procedure in case the evaluation of the filling volume is based on weak pressure signals and thus potentially unreliable pressure samples.

The method is typically a computer-implemented method performed by a computer upon execution of a computer program. Consequently, according to another aspect of the disclosure, there is provided a computer program for automatic evaluation of a filling volume of an oesophageal balloon catheter inserted into a mechanically ventilated patient. The computer program comprises computer-readable instructions, which when executed by a processor of the computer, causes the computer to obtain samples of an airway pressure (P_{aw}) and an oesophageal pressure (Pₑₛ) of a mechanically ventilated patient during an occlusion period in which respiration of the patient is prevented, evaluate the filling volume of the oesophageal balloon catheter by determining a ratio (ΔPₑₛ/ΔP_{aw}) between Pₑₛ and P_{aw} from a regression analysis of the obtained Pₑₛ and P_{aw} samples, and communicate a result of the evaluation to a user, e.g. to an operator of a breathing apparatus providing the mechanical ventilation to the patient.

The computer program may further comprise instructions for causing the computer to perform any of, or any combination of, the above described method steps.

According to another aspect of the disclosure, there is provided a computer program product comprising a non-transitory computer-readable storage medium storing the computer program. The storage medium may e.g. be a non-transitory memory hardware device of the computer on which the computer program is run.

The computer may be a stand-alone computer or a computer residing in any type of medical equipment for automatic evaluation of a filling volume of an oesophageal balloon catheter inserted into a mechanically ventilated patient. For example, the computer may be a computer of the breathing apparatus providing the mechanical ventilation to the patient, or a computer of a patient monitoring system for monitoring the patient and/or the mechanical ventilation of the patient.

Consequently, according to yet another aspect of the disclosure, there is provided a computerized system for automatic evaluation of a filling volume of an oesophageal balloon catheter inserted into a mechanically ventilated patient. The system comprises a first pressure sensor for obtaining samples of an airway pressure, P_{aw}, of the patient during an occlusion period in which respiration of the patient is prevented, a second pressure sensor for obtaining samples of an oesophageal pressure, Pₑₛ, of the patient during the occlusion period, and a computer for processing the Pₑₛ and P_{aw} samples. The computer is configured to evaluate the filling volume of the oesophageal balloon catheter by determining a ratio, ΔPₑₛ/ΔP_{aw}, between Pₑₛ and P_{aw} from a regression analysis of the Pₑₛ and P_{aw} samples, and to cause a result of the evaluation to be communicated to a user.

The computer may be configured to determine the ΔPₑₛ/ΔP_{aw} ratio as a slope of a curve resulting from the regression analysis, for example as a slope of a linear curve resulting from a linear regression analysis of the Pₑₛ and P_{aw} samples.

The computer may further be configured to determine, based on the ΔPₑₛ/ΔP_{aw} ratio, if the filling volume of the oesophageal balloon catheter is within a predetermined acceptance range, and to cause information on whether or not the filling volume of the oesophageal balloon catheter is within the acceptance range to be communicated to the user.

The computer may further be configured to determine a quality measure of the evaluation based on a correlation between the Pₑₛ and P_{aw} samples, and to cause information indicative of an uncertainty in the evaluation of the filling volume of the oesophageal balloon catheter to be communicated to the user, which information is based on the determined quality measure. The quality measure may for instance be the coefficient of determination, R², of the regression analysis.

The computer may further be configured to determine a magnitude of change in Pₑₛ and/or P_{aw} during the occlusion period from the obtained Pₑₛ and P_{aw} samples, and cause information comprising a recommendation to repeat the evaluation of the filling volume of oesophageal balloon catheter to be communicated to the user if the magnitude of change in Pₑₛ and/or P_{aw} during the occlusion period is below a certain threshold value.

More advantageous aspects of the proposed method, computer program and system for automatic evaluation of the filling volume of an oesophageal balloon catheter will be described in the detailed description of embodiments following hereinafter.

### BRIEF DESCRIPTION OF THE DRAWINGS

The present invention will become more fully understood from the detailed description provided hereinafter and the accompanying drawings which are given by way of illustration only. In the different drawings, same reference numerals correspond to the same element.

FIG. 1 illustrates an example of a system for automatic evaluation of a filling volume of an oesophageal balloon catheter inserted into a mechanically ventilated patient.

FIG. 2 is a flow chart illustrating an example of a method for automatic evaluation of a filling volume of an oesophageal balloon catheter.

FIGS. 3A-6B illustrate different scenarios in which oesophageal pressure and airway pressure curves and samples are obtained during an occlusion period, as well as the result of a regression analysis performed on the samples obtained in the different scenarios.

### DETAILED DESCRIPTION

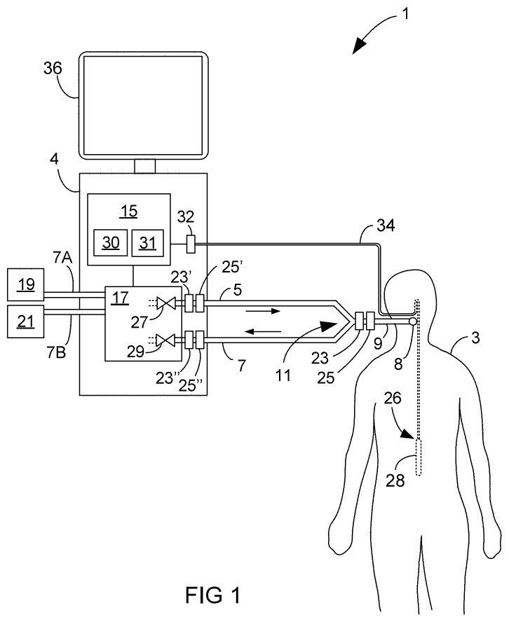

FIG. 1 illustrates an exemplary embodiment of a system 1 for automatic evaluation of a filling volume of an oesophageal balloon catheter inserted into a mechanically ventilated patient 3. The system comprises a breathing apparatus 4 for mechanically ventilating the patient 3. The breathing apparatus 4 may be any type of apparatus capable of providing mechanical ventilation to the patient 3 through the supply of pressurised breathing gas to the airways of the patient. Ventilators and anaesthesia machines are non-limiting examples of such breathing apparatuses.

The breathing apparatus 4 is connected to the patient 3 via a patient circuit comprising an inspiratory line 5 for supplying breathing gas to the patient 3, and an expiratory line 7 for conveying expiration gas away from the patient 3. The inspiratory line 5 and the expiratory line 7 are connected to the patient 3 via a patient connector 8, such as an endotracheal tube or a face mask. The inspiratory line 5 and the expiratory line 7 may be connected to the patient connector 8 either directly (if using double lumen tubing) or via a Y-piece. In the illustrated example, the inspiratory line 5 and the expiratory line 7 are connected to a common line 9 via a Y-piece 11, which common line 9 is connected to the patient 3 via the patient connector 8.

The breathing apparatus 4 comprises a control unit or control computer 15 for controlling the ventilation of the patient 3 based on pre-set parameters and/or measurements obtained by various sensors of the breathing apparatus. The control computer 15 controls the ventilation of the patient 3 by controlling a pneumatic unit 17 of the breathing apparatus 2, which pneumatic unit 17 is connected on one hand to one or more gas sources 19, 21 and on the other hand to the inspiratory line 5 for regulating a flow and/or pressure of breathing gas delivered to the patient 3. The pneumatic unit 17 may comprise various gas mixing and regulating means well known in the art of ventilation, such as gas mixing chambers, controllable gas mixing valves, turbines, controllable inspiration and/or expiration valves, etc.

The system 1 further comprises one or more flow sensors 23, 23′, 23″ for measuring respiratory flow, and one or more pressure sensors 25, 25′, 25″ for measuring respiratory pressure. The flow sensor 23 may be a proximal flow sensor located close to the patient 3 (e.g. in or close to the Y-piece 11) and configured to measure both an inspiratory flow of breathing gas delivered towards the patient 3 during inspiration, and an expiratory flow of gas exhaled by the patient 3 during expiration. Likewise, the pressure sensor 25 may be a proximal pressure sensor located close to the patient 3 (e.g. in or close to the Y-piece 11) and configured to measure, during both inspiration and expiration, a proximate patient pressure substantially corresponding to an airway pressure of the patient 3. Alternatively or in addition to the flow sensor 23 and the pressure sensor 25, the breathing apparatus 4 may comprise one or more internal flow sensors for measuring respiratory gas flow, and/or one or more internal pressure sensors for measuring respiratory gas pressure. For example, the breathing apparatus 4 may comprise a flow sensor 23′ for measuring a flow of breathing gas in an inspiratory flow channel of the breathing apparatus 4, and/or a pressure sensor 25′ for measuring a gas pressure in the inspiratory flow channel of the breathing apparatus. Alternatively, or in addition, the breathing apparatus 4 may comprise a flow sensor 23″ for measuring a flow of expiration gas in an expiratory flow channel of the breathing apparatus 2, and/or a pressure sensor 25″ for measuring a gas pressure in the expiratory flow channel of the breathing apparatus.

The measurement signals obtained by the one or more flow sensors 23, 23′, 23″ and the one or more pressure sensors 25, 25′, 25″ are transmitted to the control computer 15 of the breathing apparatus 4, whereby the control computer 15 can control the flow and volume of breathing gas delivered to the patient 3, as well as the airway pressure of the patient 3, by controlling the pneumatic unit 17 based on the measurement signals. In this exemplary embodiment, the pneumatic unit 17 comprises a controllable inspiratory valve 27 for regulating inspiratory flow and pressure, and a controllable expiratory valve 29 for controlling an expiratory pressure applied to the patient 3 during expiration, including a positive end-expiratory pressure (PEEP) of the patient.

The system 1 further comprises an oesophageal pressure sensor arrangement for measuring an oesophageal pressure of the patient. The oesophageal pressure sensor arrangement comprises an oesophageal balloon catheter 26 including an oesophageal balloon 28 intended to be inserted into the oesophagus of the patient 3 during mechanical ventilation of the patient. The oesophageal pressure sensor arrangement further comprises a pressure sensor 32 that is arranged in fluid communication with the oesophageal balloon 28 via a pressure extension tube 34. The oesophageal balloon 28 and the pressure extension tube 34 are filled with a fluid, typically air. Changes in the oesophageal pressure of the patient 3 causes compression or expansion of the balloon 28, which compression or expansion affects the fluid pressure in the pressure extension tube 34. The fluid pressure is measured by the pressure sensor 32 and used by the control computer 15 to determine the oesophageal pressure of the patient 3.

In this example, the pressure sensor 32 forms part of the breathing apparatus 4. In other examples, the pressure sensor 32 may form part of the oesophageal balloon catheter 26, whereby the pressure sensor may be configured to communicate the pressure measurements to the control computer 15 of the breathing apparatus via a signalling line for electronic communication between the oesophageal balloon catheter 26 and the breathing apparatus 4.

As discussed above, the oesophageal pressure of the patient 3 may be used as a surrogate for pleural pressure and thus provide useful information on the chest wall mechanics of the ventilated patient 3. For example, the control computer 15 may be configured to determine a transpulmonary pressure of the ventilated patient 3 from oesophageal and airway pressure measurements, and to communicate information relating to the transpulmonary pressure to an operator of the breathing apparatus 4. The control computer 15 may also be configured to use the oesophageal pressure measurements in addition to the respiratory flow and/or pressure measurements to control the flow and volume of breathing gas delivered to the patient 3, as well as the airway pressure of the patient 3, by controlling the pneumatic unit 17 based on the oesophageal pressure measurements. For example, the control computer 15 may be configured to suggest or automatically select ventilation settings that are adapted to the pulmonary mechanics of the patient 3, taking both lung and chest wall compliance into consideration. In particular, the additional information on the chest wall mechanics of the ventilated patient 3, provided to the control computer 15 via the oesophageal pressure measurements, may be used by the control computer 15 in lung recruitability assessments, lung recruitment manoeuvres, and in the adjustment of ventilation parameters, such as PEEP and tidal volume.

The volume of fluid within the oesophageal balloon catheter 26, i.e. the filling volume of the oesophageal balloon catheter 26, is crucial to the accuracy in oesophageal pressure determination. Too small or too large filling volumes result in unreliable oesophageal pressure measurements, often deviating from the actual oesophageal pressure of the patient in an unpredictable manner that cannot easily be compensated for.

The control computer 15 comprises a processor or processing unit 30 and a non-volatile memory hardware device 31 storing one or more computer programs for controlling the operation of the breathing apparatus 4, including a computer program for automatic evaluation of the filling volume of the oesophageal balloon catheter 26. The computer program for automatic evaluation of the filling volume of the oesophageal balloon catheter 26 can be initiated by an operator of the breathing apparatus 4, e.g. by actuating a touch-button of a graphical user interface (GUI) displayed on a display 36 of the breathing apparatus 4.

Upon initiation of the computer program, the system 1 will perform a fully automatic evaluation of the filling volume of the oesophageal balloon catheter and present a result of the evaluation to the operator, e.g. in form of a confirmation of correct filling volume or an alert for making the operator aware of incorrect filling volume, displayed on the display of the breathing apparatus 4.

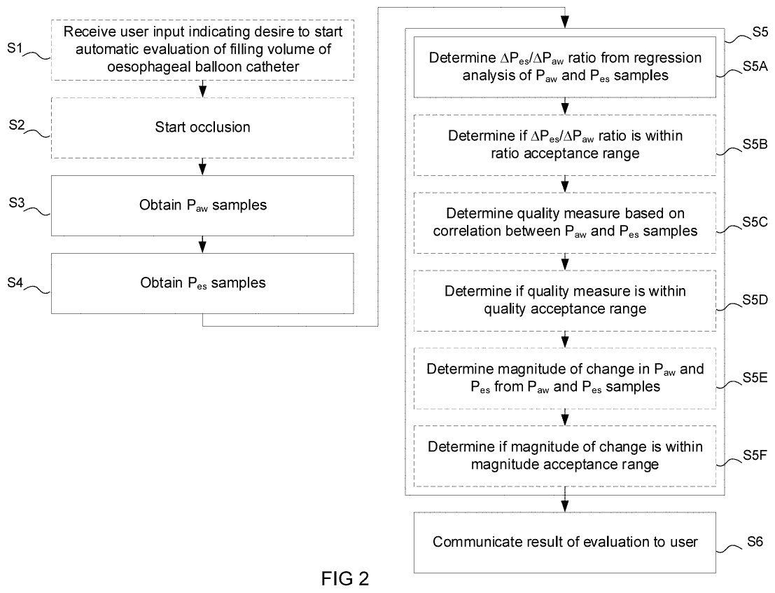

The evaluation process will now be described with reference to the flowchart shown in FIG. 2, illustrating a method for automatic evaluation of a filling volume of an oesophageal balloon catheter 26 according to an exemplary embodiment of the present disclosure. When describing the method, simultaneous reference will be made to the system 1 and system components illustrated in FIG. 1. Unless stated otherwise, any actions and method steps described hereinafter are performed by, or caused by, the control computer 15 of the breathing apparatus 4 upon execution by the processing unit 30 of different code segments of the computer program for automatic evaluation of the filling volume of the oesophageal balloon catheter 2, stored in the memory 31.

In a first optional step, S1, user input indicating a desire to start an automatic evaluation of the filling volume of the oesophageal balloon catheter 26 is received. The user input may be received via any type of user input means of the system 1, for example via a touch screen of the display 36.

In a second optional step, S2, an occlusion period in which respiration of the patient 3 is prevented is initiated. During occlusion, gas flow to and from the patient 3 is prevented. Occlusion may be achieved by the control computer 15 causing the inspiratory valve 27 and the expiratory valve 29 of the breathing apparatus 4 to close, and to be kept closed during the duration of the occlusion period. The duration of the occlusion period may be predetermined. The duration of the occlusion period may be in the range of 5-15 seconds, and preferably approximately 10 seconds. The occlusion may be an end-expiratory occlusion, meaning that the occlusion is initiated at the end of an expiratory phase.

In a third step, S3, occurring during the occlusion period, samples of the airway pressure, P_{aw}, of the patient 3 are obtained. The P_{aw} samples may, for instance, be collected by the proximal pressure sensor 25 situated in or close to the Y-piece 11 of the patient circuit, or calculated by the control computer 15 based on pressure samples obtained by the pressure sensors 25′ and 25″ situated in the inspiratory and expiratory flow channels of the breathing apparatus 4.

In a fourth step, S4, also occurring during the occlusion period, samples of the oesophageal pressure, Pₑₛ, of the patient 3 are obtained. The Pₑₛ samples are obtained by the oesophageal pressure sensor arrangement comprising the oesophageal balloon catheter 26.

The P_{aw} and Pₑₛ samples should be obtained during a period in which P_{aw} and Pₑₛ of the patient 3 varies, at least to some extent. Therefore, in passive patients having no spontaneous breathing activity, the method may comprise an additional step of manually compressing the rib cage of the patient 3 during the occlusion period in order to produce variations in P_{aw} and Pₑₛ. As described above, a procedure for evaluating the filling volume of an oesophageal balloon catheter by studying the relation between P_{aw} and Pₑₛ during an occlusion period in which variations in P_{aw} and Pₑₛ are caused by manual compression of the patient's rib cage is sometimes referred to as a positive pressure occlusion test. The rib cage of the patient may, for instance, be manually compressed 2-6 times during the occlusion period, and preferably about 4 times.

In active patients having a spontaneous breathing activity, no manual compression of the rib cage is generally needed. Instead, spontaneous breathing attempts by the patient 3 during the occlusion period generates the required variations in P_{aw} and Pₑₛ. As described above, a procedure for evaluating the filling volume of an oesophageal balloon catheter by studying the relation between P_{aw} and Pₑₛ during an occlusion period in which variations in the airway pressure and oesophageal pressure are caused by spontaneous breathing attempts by the patient is sometimes referred to as a Baydur occlusion test.

For reliable evaluation of the filling volume of the oesophageal balloon catheter, it is important for the sample size of the P_{aw} and Pₑₛ samples to be big enough. Therefore, sampling of P_{aw} and Pₑₛ should be performed during a sufficiently long period of time, at a sufficiently high sampling frequency. The sample size should preferably be at least 50, more preferably at least 100, and most preferably at least 500. Preferably, P_{aw} and Pₑₛ samples are obtained during substantially the entire occlusion period at a sampling frequency of 10 Hz or more. Preferably, the sampling frequency is about 100 Hz. In one exemplary embodiment, P_{aw} and Pₑₛ samples are obtained during substantially the entire duration of an occlusion period of 10 seconds, at a sampling frequency of 100 Hz, resulting in a sample size of approximately 1000.

In a fifth step, S5, the filling volume of the oesophageal balloon catheter 26 is evaluated based on the Pₑₛ and P_{aw} samples obtained during the occlusion period.

This is achieved by performing, in a first evaluation step S5A, a regression analysis of the Pₑₛ and P_{aw} samples, and determining a ratio, ΔPₑₛ/ΔP_{aw}, between Pₑₛ and P_{aw} from the regression analysis. The ΔPₑₛ/ΔP_{aw} ratio may be determined using any type of automated regression analysis for estimating a relationship between Pₑₛ and P_{aw}. For example, the control computer 15 may be configured to determine the ΔPₑₛ/ΔP_{aw} ratio as a slope of a curve resulting from the regression analysis, i.e. as the slope of a regression function estimated from the regression analysis. The regression analysis may be a linear regression analysis assuming a linear relationship between Pₑₛ and P_{aw}. In this case, the ΔPₑₛ/ΔP_{aw} ratio may be determined as the slope of the linear regression function estimated from the linear regression analysis.

For example, the linear regression analysis of Pₑₛ and P_{aw} samples may be based on an assumption of a linear regression function expressing the relationship between Pₑₛ and P_{aw} as:

Pₑₛ=a+b·Paw,

where Pes is the oesophageal pressure of the ventilated subject, Paw is the airway pressure of the ventilated subject, and a and b are coefficients that can be determined e.g. using the least square error optimization technique. The coefficient b is the slope of the linear regression function and represents the ΔPₑₛ/ΔP_{aw} ratio.

In other embodiments, the regression analysis may be a non-linear regression analysis and the ΔPₑₛ/ΔP_{aw} ratio may be determined based on a non-linear regression function estimated from the regression analysis.

The evaluation may further comprise a second evaluation step, S5B, of determining whether the ΔPₑₛ/ΔP_{aw} ratio is within a predetermined ratio acceptance range. The predetermined ratio acceptance range may be e.g. 0.6-1.4 or, more preferably, 0.8-1.2. If the ΔPₑₛ/ΔP_{aw} ratio is within the predetermined ratio acceptance range, the filling volume of the oesophageal balloon catheter 26 is considered to be within a filling volume acceptance range. In this case, the oesophageal balloon catheter 26 is deemed to be capable of obtaining accurate and reliable measurements of the oesophageal pressure of the ventilated patient 3. If, on the other hand, the ΔPₑₛ/ΔP_{aw} ratio is outside the predetermined ratio acceptance range, the filling volume of the oesophageal balloon catheter is considered to be outside the filling volume acceptance range. In this case, the oesophageal catheter 26 is deemed to be incapable of obtaining accurate and reliable measurements of the oesophageal pressure of the ventilated patient 3.

The evaluation may further comprise a third evaluation step, S5C, in which a quality measure of the determination of the ΔPₑₛ/ΔP_{aw} ratio is determined based on a correlation between the Pₑₛ and P_{aw} samples. The quality measure may be any measure indicative of how well the regression function estimated in step S5A approximates the obtained Pₑₛ and P_{aw} samples. In one example, the quality measure may be the coefficient of determination, normally referred to as the R² coefficient.

As understood by a skilled person, the R² coefficient may in this instance be calculated e.g. from the following relations:

$${R}^{2} = 1 - \frac{{S}_{r e s}}{{S}_{t o t}} , {S}_{r e s} = {\sum }_{i} {( {Pes}_{i} - PesFi {t}_{i} )}^{2} , {S}_{t o t} = {\sum }_{i} {( {Pes}_{i} - {Pes}_{m e a n} )}^{2} ,$$

where Pesᵢ is Pₑₛ sample number i for i=1 to N, where N is the total number of Pₑₛ samples, PesFitᵢ is a predicted value of Pest, calculated from P_{aw} sample number i and the assumed linear relationship between Pₑₛ and P_{aw} expressed by the regression function, Pesₘₑₐₙ is the mean of the Pₑₛ samples, Sᵣₑₛ is the residual sum of squares, and Sₜₒₜ is the total sum of squares.

The evaluation may further comprise a fourth evaluation step, S5D, of determining whether the quality measure determined in step S5C is within a predetermined quality acceptance range. For example, if the quality measure is the R² coefficient, the quality measure may be deemed to be within the quality acceptance range if R²>0.7 or, more preferably, if R²>0.9.

The evaluation may further comprise a fifth evaluation step, S5E, in which at least one of a magnitude of change in Pₑₛ and a magnitude of change in P_{aw} during the occlusion period is determined from the obtained Pₑₛ and P_{aw} samples.

The evaluation may further comprise a sixth evaluation step, S5F, of determining whether the at least one magnitude of change determined in step S5C is within a predetermined magnitude acceptance range. Preferably, the step involves determining whether each of the magnitude of change in Pₑₛ and the magnitude of change in P_{aw} during the occlusion period is within the predetermined magnitude acceptance range. The predetermined magnitude acceptance range may be defined by a minimum threshold value for the change in magnitude of any or both of Pₑₛ and P_{aw}. For example, if the magnitude of change in any of Pₑₛ or P_{aw} during the occlusion period is less than 2 cmH2O, the magnitude of change may be deemed to be outside the magnitude acceptance range.

After the evaluation, the method continuous to step S6 in which a result of the evaluation of the filling volume of the oesophageal balloon catheter 26 is communicated to a user, e.g. to an operator of the breathing apparatus 4.

The control computer 15 may cause the result of the evaluation to be communicated to the user in different ways. For example, the result may be visually communicated to the user via a display of the system 1, such as the display 36 of the breathing apparatus 4, or aurally communicated to the user via one or more loudspeakers of the system 1.

The result of the evaluation may comprise the ΔPₑₛ/ΔP_{aw} ratio determined in step S5A. Communicating the ΔPₑₛ/ΔP_{aw} ratio to a trained clinician allows the clinician to decide on whether the filling volume of the oesophageal balloon catheter 36 is accurate enough to provide for reliable measurements of the oesophageal pressure of the ventilated patient 3. Instead of, or in addition to, the determined ΔPₑₛ/ΔP_{aw} ratio, the result that is communicated to the user may comprise an indication on whether or not the filling volume of the oesophageal balloon catheter is acceptable. This allows the clinician to take appropriate actions (e.g. replacing or refilling the oesophageal balloon catheter) without extensive knowledge on the relationship between the ΔPₑₛ/ΔP_{aw} ratio and the filling volume of the oesophageal balloon catheter 26. The indication is typically based on the determined ΔPₑₛ/ΔP_{aw} ratio but does not necessarily include the numeric value of the ΔPₑₛ/ΔP_{aw} ratio. For example, the result may comprise an indication indicating whether or not the determined ΔPₑₛ/ΔP_{aw} ratio is within the ratio acceptance range, as determined in step S5B. The indication may comprise a first symbol (e.g. a green symbol) that is displayed on the display 36 if the ΔPₑₛ/ΔP_{aw} ratio is within the ratio acceptance range, and a second and different symbol (e.g. a red symbol) that is displayed on the display 36 if the ΔPₑₛ/ΔP_{aw} ratio is outside the ratio acceptance range.

The result of the evaluation may further comprise a recommendation to the user to adjust the filling volume of the oesophageal catheter 26. For instance, the result may comprise a recommendation to the user to adjust the filling volume of the oesophageal catheter 26 if the ΔPₑₛ/ΔP_{aw} ratio is outside the ratio acceptance range, as determined in step S5B. The recommendation may be communicated to the user by the control computer 15 causing the recommendation to be displayed on the display 36. Typically, if the ΔPₑₛ/ΔP_{aw} ratio is outside the ratio acceptance range, the filling volume of the oesophageal balloon catheter 26 may be assumed to be too small, wherefore the recommendation in this case may comprise a recommendation to refill the oesophageal balloon catheter 26.

The result of the evaluation may further comprise information indicative of the uncertainty in the determination of the ΔPₑₛ/ΔP_{aw} ratio. This information may be based on the quality measure determined in step S5C. For instance, the information may be based on whether or not the quality measure is within a predetermined quality acceptance range, as determined in step S5D. If the quality measure is outside the quality acceptance range, the information may comprise any or both of an alert informing the user of high uncertainty in the evaluation of the filling volume of the oesophageal balloon catheter, and a recommendation to repeat the evaluation.

The result of the evaluation may further comprise information relating to the variations in Pₑₛ and/or P_{aw} during the occlusion period, i.e. information relating to the magnitude of change in any or both of Pₑₛ and P_{aw} during the occlusion period, as determined in step S5E. The information may, for instance, be based on whether or not the magnitude of change in any or both of Pₑₛ and P_{aw} is outside the predetermined magnitude acceptance range, as determined in step S5F. If any or both of the magnitude of change in Pₑₛ and P_{aw} is outside the magnitude acceptance range, the information may, for instance, comprise and alert informing the user of weak pressure signals during evaluation and/or a recommendation to repeat evaluation due to weak pressure signals.

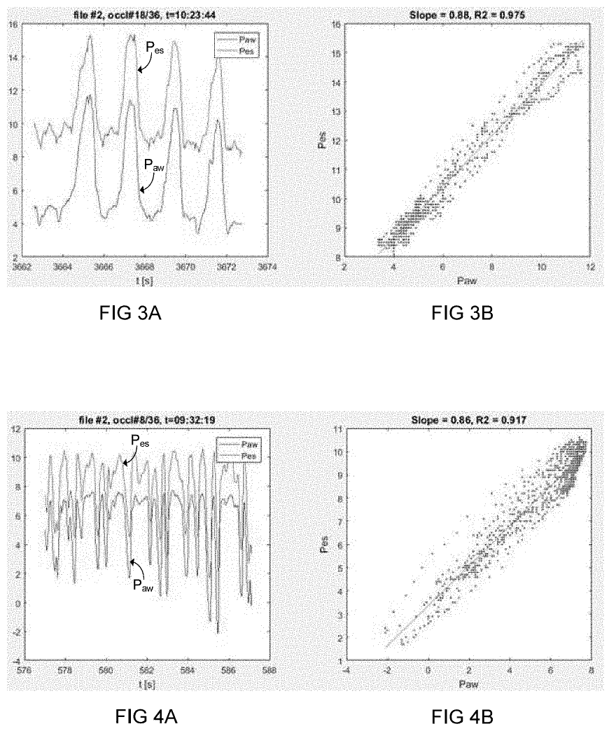

FIGS. 3A-6B illustrate the proposed method in terms of four examples of data sets obtained through sampling of Pₑₛ and P_{aw} during occlusion.

FIG. 3A illustrates variations in Pₑₛ (upper graph) and P_{aw} (lower graph) during a 10 s occlusion test with four chest compressions on an inactive patient, and FIG. 3B illustrates a linear regression analysis performed on Pₑₛ and P_{aw} samples obtained during the 10 s occlusion test at a sampling frequency of 100 Hz. In FIG. 3B, each dot represents a Pₑₛ-P_{aw} sample and the curve represents a regression function estimated from the Pₑₛ-P_{aw} samples. The slope of the regression function corresponds to the ΔPₑₛ/ΔP_{aw} ratio. In this example, the slope is 0.88, corresponding to a ΔPₑₛ/ΔP_{aw} ratio which is well within the above mentioned example of a predetermined ratio acceptance range. Calculating from the Pₑₛ-P_{aw} samples the coefficient of determination as a quality measure of the determination of the ΔPₑₛ/ΔP_{aw} ratio in accordance with the above described principles results in an R² value of 0.975, which is well within the above mentioned example of a predetermined quality acceptance range. Accordingly, FIGS. 3A-3B illustrate a scenario in which the proposed method for automatic evaluation of the filling volume of an oesophageal balloon catheter would confirm correct filling volume of the oesophageal balloon catheter with a high degree of certainty.

FIG. 4A illustrates variations in Pₑₛ (upper graph) and P_{aw} (lower graph) during a 10 s occlusion test with a sequence of several breathing attempts made by an active patient, and FIG. 4B illustrates a linear regression analysis performed on the Pₑₛ and P_{aw} samples obtained during the 10 s occlusion test at a sampling frequency of 100 Hz. In this scenario, the slope of the regression function is 0.86 and the R² coefficient is 0.917, indicating that the proposed method would confirm correct filling volume of the oesophageal balloon catheter with a high degree of certainty also in this situation. In contrast, as understood from FIG. 4A, manual evaluation of the filling volume of the oesophageal balloon catheter from ocular identification of maximum and minimum pressure curve values would be a challenging task associated with a high degree of uncertainty.

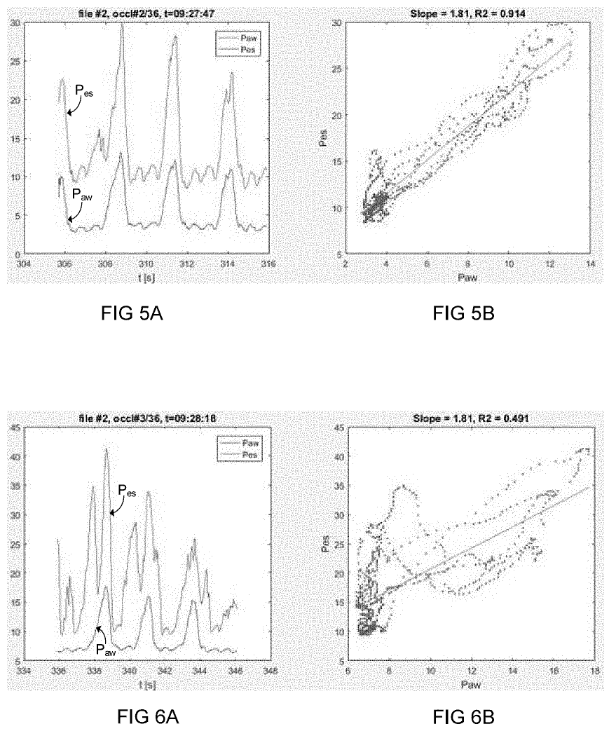

FIG. 5A illustrates variations in Pₑₛ (upper graph) and P_{aw} (lower graph) during a 10 s occlusion test with four chest compressions on an inactive patient, and FIG. 5B illustrates a linear regression analysis performed on the Pₑₛ and P_{aw} samples obtained during the 10 s occlusion test at a sampling frequency of 100 Hz. In this scenario, the slope of the regression function is 1.81 and the R² coefficient is 0.914. A ΔPₑₛ/ΔP_{aw} ratio (corresponding to the slope of the linear regression function) of 1.81 is outside the exemplary ratio acceptance range discussed above, whereas an R² coefficient of 0.914 is well within the exemplary quality acceptance range discussed above. Accordingly, FIGS. 5A-5B illustrate a scenario in which the proposed method for automatic evaluation of the filling volume of an oesophageal balloon catheter would confirm incorrect filling volume of the oesophageal balloon catheter with a high degree of certainty.

FIG. 6A illustrates variations in Pₑₛ (upper graph) and P_{aw} (lower graph) during a 10 s occlusion test with a sequence of several breathing attempts made by an active patient, and FIG. 6B illustrates a linear regression analysis performed on the Pₑₛ and P_{aw} samples obtained during the 10 s occlusion test at a sampling frequency of 100 Hz. In this scenario, the slope of the regression function is 1.81 and the R² coefficient is 0.491. An R² coefficient of 0.491 is outside the above mentioned example of a quality measure acceptance range and indicates that the data is contaminated by large disturbances. Accordingly, FIGS. 6A-6B illustrate a scenario in which the proposed method for automatic evaluation of the filling volume of an oesophageal balloon catheter could not evaluate the filling volume of the oesophageal balloon catheter with a satisfactory degree of certainty. As described above, this could, for instance, cause a recommendation to repeat the evaluation to be communicated to the user.

1-15. (canceled)

16. A method for automatic evaluation of a filling volume of an oesophageal balloon catheter inserted into a mechanically ventilated patient, comprising:
obtaining samples of an airway pressure, P_{aw}, of the patient during an occlusion period in which respiration of the patient is prevented;
obtaining samples of an oesophageal pressure, Pₑₛ, of the patient during the occlusion period;
evaluating the filling volume of the oesophageal balloon catheter by determining a ratio, ΔPₑₛ/ΔP_{aw}, between Pₑₛ and P_{aw} from a regression analysis of the Pₑₛ and P_{aw} samples; and
communicating a result of the evaluation to a user.

17. The method of claim 16, further comprising:
determining, based on the ΔPₑₛ/ΔP_{aw} ratio, if the filling volume of the oesophageal balloon catheter is within a predetermined acceptance range; and
communicating whether or not the filling volume of the oesophageal balloon catheter is within the acceptance range to the user.

18. The method of claim 16, further comprising:
determining a quality measure of the evaluation based on a correlation between the Pₑₛ and P_{aw} samples; and
communicating information indicative of an uncertainty in the evaluation of the filling volume of the oesophageal balloon catheter to the user, which information is based on the determined quality measure.

19. The method of claim 16, further comprising:
determining a magnitude of change in Pₑₛ and/or P_{aw} during the occlusion period from the obtained Pₑₛ and P_{aw} samples; and
communicating information comprising a recommendation to repeat the evaluation of the filling volume of the oesophageal balloon catheter to the user if the magnitude of change in Pₑₛ and/or P_{aw} during the occlusion period is below a certain threshold value.

20. The method of claim 16, wherein the ΔPₑₛ/ΔP_{aw} ratio is determined as a slope of a curve resulting from the regression analysis.

21. The method of claim 16, wherein the ΔPₑₛ/ΔP_{aw} ratio is determined as a slope of a linear curve resulting from a linear regression analysis of the Pₑₛ and P_{aw} samples.

22. The method of claim 18, wherein the quality measure is the coefficient of determination, R², of the regression analysis.

23. A computer program for automatic evaluation of a filling volume of an oesophageal balloon catheter inserted into a mechanically ventilated patient, the computer program comprising computer-readable instructions which, when executed by a computer, causes the computer to perform the method according to claim 16.

24. A system for automatic evaluation of a filling volume of an oesophageal balloon catheter inserted into a mechanically ventilated patient, comprising:
a first pressure sensor obtaining samples of an airway pressure, P_{aw}, of the patient during an occlusion period in which respiration of the patient is prevented;
a second pressure sensor obtaining samples of an oesophageal pressure, Pₑₛ, of the patient during the occlusion period; and
a computer processing the Pₑₛ and P_{aw} samples,
wherein the computer is configured to
evaluate the filling volume of the oesophageal balloon catheter by determining a ratio, ΔPₑₛ/ΔP_{aw}, between Pₑₛ and P_{aw} from a regression analysis of the Pₑₛ and P_{aw} samples, and
cause a result of the evaluation to be communicated to a user.

25. The system of claim 24, wherein the computer is configured to
determine, based on the ΔPₑₛ/ΔP_{aw} ratio, if the filling volume of the oesophageal balloon catheter (26) is within a predetermined acceptance range, and
cause information on whether or not the filling volume of the oesophageal balloon catheter is within the acceptance range to be communicated to the user.

26. The system of claim 24, wherein the computer further is configured to
determine a quality measure of the evaluation based on a correlation between the Pₑₛ and P_{aw} samples, and
cause information indicative of an uncertainty in the evaluation of the filling volume of the oesophageal balloon catheter to be communicated to the user, which information is based on the determined quality measure.

27. The system of claim 24, wherein the computer further is configured to
determine a magnitude of change in Pₑₛ and/or P_{aw} during the occlusion period from the obtained Pₑₛ and P_{aw} samples, and
cause information comprising a recommendation to repeat the evaluation of the filling volume of oesophageal balloon catheter to be communicated to the user if the magnitude of change in Pₑₛ and/or P_{aw} during the occlusion period is below a certain threshold value.

28. The system of claim 24, wherein the computer is configured to determine the ΔPₑₛ/ΔP_{aw} ratio as a slope of a curve resulting from the regression analysis.

29. The system of claim 24, wherein the computer is configured to determine the ΔPₑₛ/ΔP_{aw} ratio as a slope of a linear curve resulting from a linear regression analysis of the Pₑₛ and P_{aw} samples.

30. The system of claim 26, wherein the quality measure is the coefficient of determination, R², of the regression analysis.
